(19) United States
(12) Patent Application Publication
(10) Pub. No.: US 2005/0109087 A1
(43) Pub. Date: May 26, 2005

(54) Methods and compositions for determining the sources of fluids or particulates from subterranean formations

(76) Inventors: Ian D. Robb, Duncan, OK (US); Robert E. Hanes JR., Duncan, OK (US); Billy F. Slabaugh, Duncan, OK (US); Jim D. Weaver, Duncan, OK (US); Philip D. Nguyen, Duncan, OK (US); Johnny A. Barton, Marlow, OK (US)

Correspondence Address: Robert A. Kent;Halliburton Energy Services, 2600 S. 2nd Street, Duncan, OK, 73536-0440, US

(21) Appl. No.: 10/723,418

(22) Filed: Nov. 25, 2003

(51) Int. Cl. G01N 33/487

(52) U.S. Cl. 73/53.01

(57) ABSTRACT

The present invention relates to methods of determining the source of a particular fluid or particulate, and to novel tracer matrixes that may be used in such methods. In one embodiment, the present invention provides a method of detecting particulate or fluid flow from a first location to a second location comprising the steps of: introducing a tracer matrix comprising a polymeric material and a photoactive material to the first location; and detecting the photoactive material at the second location. In another embodiment, the present invention provides a method of detecting flow from a first zone and a second zone in a multizonal well in a subterranean formation comprising the steps of: introducing a first photoactive tracer into the first zone; introducing a second photoactive tracer into the second zone; and detecting the first and the second photoactive tracers in the return flow from the first and second zones.

### BACKGROUND OF THE INVENTION

1. Field of the Invention

The present invention relates to methods of determining the source of a particular fluid or particulate, and to novel tracer matrixes that may be used in such methods.

2. Description of the Related Art

In the recovery of subterranean fluids, it is often desirable to be able to identify the flow patterns of one region or location to a second region or location to determine, for example, a particular source of a particular fluid or particulate. For example, such information may be desirable in subterranean applications in the upstream energy industry where a well is plagued with particulate flowback problems; in these instances, it may be desirable to determine which particular strata are generating the flowback problem. Another example is in hydraulic fracturing operations involving multiple zone wells. In such operations, verification of flow from each zone without extensive rig costs is desirable.

The term “tracer” as used herein generally refers to a marker that is distributed in the continuous phase of a fluid, such as a subterranean fluid like a drilling, coring, drill-in, or completion fluid, that may be used to identify an item in a return flow in a desirable manner. In subterranean applications, tracers are generally retained in the treatment fluid until deposited in the formation and then produced with the fluids or particulates. Tracers also generally are measurable or detectable in small amounts. Examples of conventional tracers include radioactive isotopes or aromatic hydrocarbons, both of which often require expensive detection equipment, like high performance liquid chromatography (“HPLC”), on the return flow. Also, both of these types of conventional tracers may suffer from disadvantageous physical and chemical properties, including radioactivity and toxicity. Moreover, aromatic hydrocarbons as tracers might be indistinguishable from the reservoir hydrocarbons. Radioactive isotopes are expensive and require special handling by licensed personnel, which may lead to complex, expensive, and time-consuming processes.

### SUMMARY OF THE INVENTION

The present invention relates to methods of determining the source of a particular fluid or particulate, and to novel tracer matrixes that may be used in such methods.

In one embodiment, the present invention provides a method of detecting particulate or fluid flow from a first location to a second location comprising the steps of: introducing a tracer matrix comprising a polymeric material and a photoactive material to the first location; and detecting the photoactive material at the second location.

In another embodiment, the present invention provides a method of detecting flow from a first zone and a second zone in a multizonal well in a subterranean formation comprising the steps of: introducing a first photoactive tracer into the first zone; introducing a second photoactive tracer into the second zone; and detecting the first and the second photoactive tracers in the return flow from the first and second zones.

In another embodiment, the present invention provides a method of detecting flow in a multiple-stage hydraulic fracturing treatment comprising a plurality of stages comprising the steps of: introducing a photoactive tracer into each stage of the multiple-stage hydraulic fracturing treatment; and detecting the photoactive tracer on a return flow.

In another embodiment, the present invention provides a method of verifying the functioning of a limiting tool that limits or restricts the flow of a fluid or particulate from a first location neighboring the limiting tool to a second location comprising the steps of: introducing a photoactive tracer into the first location neighboring the limiting tool; and detecting the photoactive tracer at the second location.

In another embodiment, the present invention provides a tracer matrix composition comprising a photoactive material and a polymeric material.

In another embodiment, the present invention provides a method of making a tracer matrix that comprises a photoactive material and a polymeric material comprising the steps of: swelling the polymeric material in an organic solvent comprising a photoactive material, and removing the solvent so as to produce a tracer matrix comprising the photoactive material and the polymeric material.

The features and advantages of the present invention will be readily apparent to those skilled in the art upon a reading of the description of the preferred embodiments, which follows.

### BRIEF DESCRIPTION OF THE DRAWINGS

FIG. 1 illustrates an embodiment of the matrixes of the present invention wherein a photoactive material is distributed within a polymeric material.

FIG. 2 illustrates an embodiment of the matrixes of the present invention wherein a photoactive material is coated with a polymeric material.

### DESCRIPTION OF PREFERRED EMBODIMENTS

The present invention relates to methods of determining the source of a particular fluid or particulate, and to novel tracer matrixes that may be used in such methods.

The compositions and methods of the present invention may be useful in any application wherein it is desirable to detect the flow of, for example, a fluid or a particulate, from a first location to a second location. One example of such an instance would be detecting the flow from one stratum in a well bore to the surface. Another instance would be where it is desirable to monitor the flow of one subterranean stratum vis-a-vis other strata. In certain embodiments, the methods of the present invention may be used to determine the source of fluids or particulates from zones or strata in a multizonal well in, for example, fracturing treatments or other operations. Additionally, in certain embodiments, novel tracer matrixes described herein may be used as a means to facilitate the detection of flow of a particulate or fluid from a first location to a second location.

In some embodiments, the present invention provides methods for detecting the return flow from a multizonal well. In such methods, to determine from which zone(s) a fluid or particulate is being produced, a treatment fluid (e.g., fracturing fluid) may place a photoactive material into a zone(s) of the well. Such photoactive materials may be introduced at different stages to verify flow from different zones in a multiple-zone well. In certain preferred embodiments, each zone in the well has a different photoactive material placed in it so that each zone has a distinctive marker identifying that zone. Thus, return flow from an individual zone vis-a-vis other zones may be observed. Photoactive materials with different absorption or emitting wavelengths may provide unique “fingerprints” such that when analyzed, an identifiable “signature” for each compound, and thus each zone, may be identified. To achieve certain beneficial effects of the present invention, suitable photoactive materials should be able to withstand the subterranean conditions to which they will be subjected, e.g., they should be stable at high temperatures under conventional crosslinked fracturing fluid and breaker formulations. Photoactive materials with large extinction coefficients like fluorescein, rhodamine B, Nile Blue A, acridine orange, and the like are suitable. A preferred photoactive material comprises a fluorescein in the form of a liquid gel concentrate as opposed to a powder. Benefits in handling the liquid gel concentrate as opposed to powders may be observed. These exemplary photoactive materials may be detected in low concentrations, which helps mitigate, inter alia, the potential environmental impact of using such photoactive materials as tracers. Moreover, certain photoactive materials may be detected using ultra violet (˜254 nm) light; for example, by using a portable colorimeter in the field, which is desirable. Other suitable detection techniques also are appropriate as recognized by those skilled in the art with the benefit of this disclosure. In even more preferred embodiments, tracer matrixes of the present invention that comprise a polymeric material and a photoactive material may be used in each zone or zones in a multizonal well. A suitable detecting technique may be used to detect the photoactive material upon return flow. Quantitative as well as qualitative information may be obtained, depending on the information desired in the application.

The present invention also provides methods for detecting flow in a hydraulic fracturing stimulation treatment such as those that are performed in a multiple stage fracturing operation. In performing hydraulic fracture-stimulation treatments in multiple stages, problems may arise in identifying from which stage fluid is produced during a fluid recovery operation. Generally speaking, these multiple-stage stimulation treatments may be performed by perforating the lowest zone(s) to be fractured, performing the fracture-stimulation treatment, producing back a small amount of the treatment fluid, then isolating this zone (e.g., mechanically) while moving up the well and perforating the next zone(s) to be fractured. This operation may be repeated; oftentimes, a well may have five or more hydraulic fracturing treatments performed. Once the upper zone has been fractured, the zonal isolation can be removed, and some or all of the zones may be allowed to produce. In certain embodiments of the methods of the present invention, to identify which zones are producing zones, a suitable tracer may be pumped into the final stage of each fracturing treatment of the multiple-zone fracturing job. Examples of suitable tracers include photoactive materials with large extinction coefficients like fluorescein, rhodamine B, Nile Blue A, acridine orange, and the like. Other suitable tracers include the tracer matrixes of the present invention. If a zone is a producing zone, then its tracer will be present upon suitable detection. Each zone may comprise a different photoactive material so that each zone may have a unique marker, if desired.

In further embodiments, the present invention provides methods for verifying the functioning of tools that limit or restrict flow from one zone to another. In some cases, it may be advantageous or desirable to monitor the source of fluid or particulate flow once tools, such as valve plugs, have been activated or closed within a well bore. Those skilled in the art will recognize the advantages of having the ability to uniquely verify flow patterns and the functioning of tools without expensive testing methods. Examples of suitable tracers include photoactive materials with large extinction coefficients like fluorescein, rhodamine B, Nile Blue A, acridine orange, and the like as discussed above. Other suitable tracers include the tracer matrixes of the present invention. If a zone is a producing zone, then its tracer will be present upon detection, which may indicate, for example, whether a valve has been opened or closed.

In alternative embodiments, the present invention provides tracer matrixes that comprise a polymeric material and a photoactive material that may be used to detect fluid or particulate flow from a first location to a second location. At the second location, a suitable detector may be used to detect the presence of the photoactive material of the tracer matrix, and thereby verify the flow of the particulates or fluids of interest from the first location. This detection may involve qualitative and/or quantitative analysis, depending on the information required and the purpose for using the method.

The tracer matrixes of the present invention comprise a photoactive material and a polymeric material. Suitable photoactive materials include, but are not limited to, fluorophores, dyes, and pigments. Suitable polymeric materials include any polymeric material that can be used in conjunction with a chosen photoactive material(s) for the purposes of the present invention. Utilizing a polymeric material in conjunction with the photoactive material, inter alia, protects the photoactive material from degradation downhole due to conditions such as harsh chemical environments, high temperatures, and/or other hostile conditions that might otherwise degrade its photoactivity. Polymeric materials have demonstrated an ability to provide stability to the photoactivity of photoactive materials in hostile environments. In addition to stabilizing photoactive materials, the polymeric materials in the matrixes may help provide a relatively simple method for separating the tracer from the return flow by simple filtration.

Any methods capable of incorporating the photoactive material and the polymeric material so as to form a tracer matrix are suitable for forming the tracer matrixes of the present invention. For example, the tracer matrixes of the present invention may be covalently derivatized. Methods for covalently derivatizing commercial resin beads with photoactive materials, such as chromophores, or enclosing them into the interior of a commercial resin appear in the current literature, and therefore, can be utilized in conjunction with the present invention by one skilled in the art with the benefit of this disclosure. Known solid-phase organic synthesis techniques or solution phase techniques utilizing bond-forming reactions between a reagent or reagents to a derivatized polymeric material bead, resin, or plastic also may be suitable for use in the methods of the present invention. Techniques include, but are not limited to, nucleophilic substitution, hydroboration, organo-metallic bond-forming reactions, pericyclic bond-forming reactions, and/or combination reactions involving oxidation and/or reductions, such as reductive amination. Another method involves emulsion polymerization wherein latex particles may be synthesized to comprise photoactive material matrixes. In such instances, the degree of incorporation of the photoactive material and the latex particles is not critical as long as a sufficient amount of the photoactive material is protected so that it can provide the desired tracer functionality in a chosen application. Emulsion polymerization may be a useful process. Another method of forming the matrixes is to coat the polymeric material onto the photoactive material. This may be achieved by any suitable method including a method involving solvent evaporation and a granulation mechanism using a polymeric material, photoactive material, and a solvent. Another suitable method would be to swell a hydrophobic polymeric material in an appropriate organic solvent containing the photoactive material of choice; this may be referred to as a swelling/shrinking process. By removing the solvent through filtration, evaporation, or causing the polymeric material to shrink through the addition of a secondary solvent, the photoactive material may become hydrophobically trapped within the polymeric material such that when placed in water, the photoactive material stays substantially entrained in the polymeric material.

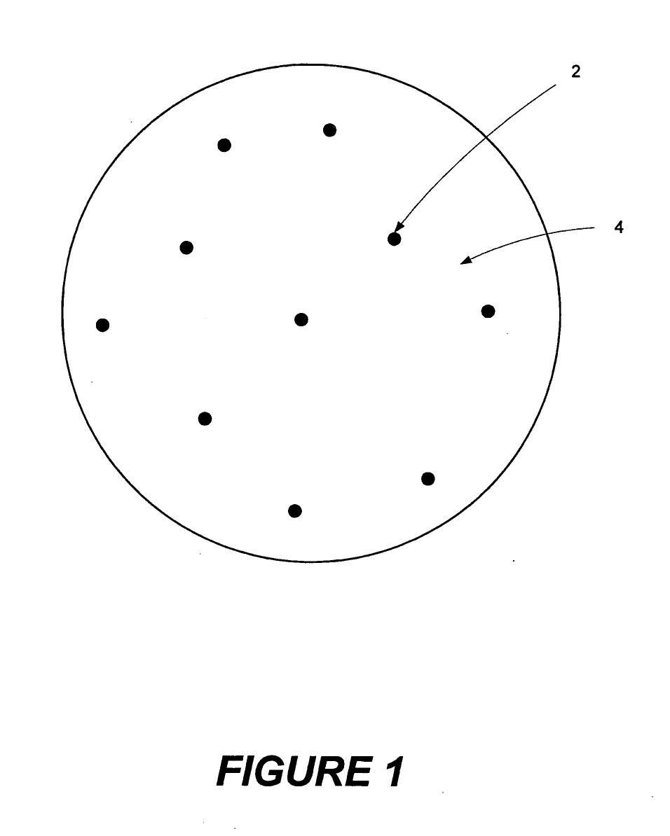

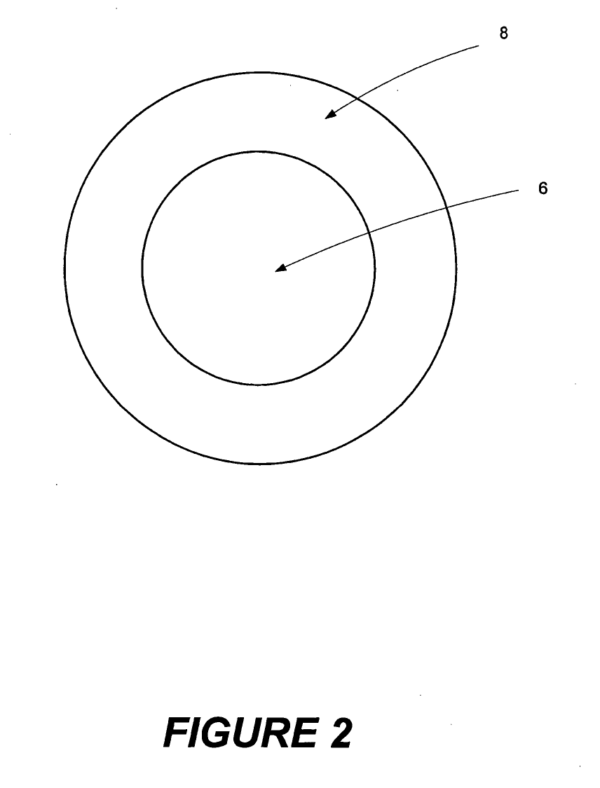

Regardless of the method utilized to form the tracer matrixes of the present invention, it is important that enough be protected so that the photoactive material acts as a suitable tracer in a chosen application. FIG. 1 illustrates one embodiment of the present invention wherein the photoactive material (2) is embedded within the polymeric material (4). FIG. 2 illustrates an embodiment wherein a photoactive particulate material (6) is coated with a polymeric material (8). These illustrations are representational only and should not be used to inappropriately limit the scope of the embodiments of the tracer matrixes of the present invention.

Suitable photoactive materials include fluorophores such as oil-soluble fluorescent materials, dyes, or pigments that can absorb or emit light in the UV, visible, or IR range of the electromagnetic spectrum. Major factors that influence one's selection of a suitable photoactive material are the emission spectrum and quantum efficiency of fluorescence (Qf), and the absorption spectrum and molar extinction coefficient (E). The product of these (Emax*Qf) can be used as a simplistic measure of relative brightness of the photoactive material. Both values (E and Qf) can be affected by, e.g., environmental factors (for instance, pH). Other factors one could consider in choosing a suitable photoactive material for a chosen application may include its relative toxicity, its ability to withstand the conditions to which it will be subjected, and its propensity to leak off into the formation. Another factor is compatibility with the detector utilized. Some field detectors can only detect certain wavelengths. In certain embodiments, a tracer should absorb or emit light at an intensity >5% to be practical in the field. In certain embodiments, photoactive materials with large extinction coefficients, for example, fluorescein and rhodamine B, offer the potential to detect these compounds in very low concentrations, which may mitigate the environmental impact of the tracer. Other examples of suitable photoactive materials include, but are not limited to, Nile Blue A, and acridine orange. In preferred embodiments, a different photoactive material is used in each zone so as to give each zone a unique fingerprint. In an alternative embodiment, a combination of photoactive materials may be used in a matrix.

Table 1 below lists suitable illustrative examples of photoactive materials that may be useful in the methods and compositions of the present invention; in no way is this an exhaustive list.

TABLE 1

| Emission | |
|---|---|
| Range | Photoactive Material |
| Blue | AMCA (aminomethylcourmarin) |
| | Cascade Blue |
| Green | Alexa 488 |
| | Cy 2 (cyanine) |
| | FITC (fluorescein isothiocyanate) |
| | FluoroGreen |
| | BODIPY FL |
| | Oregon Green 488 |
| | Oregon Green 514 |
| Yellow | Eosin |
| | Alexa 532 |
| Orange | BODIPY TMR |
| | Alexa 546 |
| | TRITC (tetrarhodamine isothiocyanate) |
| | TMR (tetramethylrhodamine) |
| Orange-red | Cy 3 (indocarbocyanine) |
| | R-phycoerythrin |
| | Lissamine rhodamine |
| | Alexa 568 |
| | Cy 3.5 |
| | Alexa 594 |
| | BODIPY TR |
| | Texas Red |
| Red-far red | Allophycocyanin |
| | Cy 5 (indodicarbocyanine) |
| | Cy 5.5 |
| | Cy 7 |

Any polymeric material that can be used in conjunction with the chosen photoactive material, inter alia, to protect the photoactive material from degradation downhole, is suitable for use in the compositions of the present invention. In certain embodiments, the polymeric material should not be soluble in water and should not be subject to degradation in the chosen application, for example, in a fracturing environment. Examples of suitable polymeric materials include, but are not limited to, latexes, polystyrenes, polyvinyl chlorides, polyesters, polyolefins, polycarbonates, and polybutadienes. Some specific examples include, but are not limited to, Wang resins (4-benzyloxybenzyl alcohol, polymer bound), Janda Jel-NH2 resins (polystyrene cross-linked with a tetrahydrofuran linker (Aldrich Chemical Company, St. Louis, Mo.)), and Merrifeld resins (chloromethylated polystyrene). Preferred cross-linked polystyrene derivatives for use in the tracer matrixes of the present invention include halomethyl, amino, or hydroxy derivatives.

Detection techniques include any technique that can measure the absorption or emission of light. Absorption spectrophotometer fluorimeters or colorimeters are suitable. For fluorophores like fluorescein and rhodamine B, a UV source (254 nm) light may be used for qualitative detection and/or a broadband detector may be used for quantitative detection. Methods for the field detection of the photoactive materials have been demonstrated through the use of hand-held calorimeters, commercially available from various sources. For instance, a Hach DR/890 colorimeter, which is available from Hach Water Treatment Chemicals, may be suitable.

Such detection techniques may provide quantitative and/or qualitative information about the tracer, depending on the information desired in the application. Qualitative determination of the presence of the tracer may be made visually or, for example, with a short, wave lamp (˜254 nm) or a field calorimeter such as a Hach DR/890. Where different tracers that have different fluorescing properties are used in different zones, Thin Layer Chromatography may be used to identify each tracer to show which zones are producing by separating the chromophores for identification on solid phase. In certain embodiments of the methods of the present invention, quantitative techniques may be used to determine how much of a tracer matrix has been recovered. Suitable techniques for such quantitative analysis include gas chromatography, HP liquid chromatography (HPLC), UV-vis spectroscopy, fluorescence spectroscopy, and mass spectroscopy. Other suitable techniques will be known to one skilled in the art with the benefit of this disclosure. For qualitative analysis, thin-layer chromatography through the separation of visible colors or visualization through UV stimulation to produce the emission of light appropriate to the chromophore may be used. In most instances, qualitative results may be sufficient, e.g., the photoactive material is there or it is not. In some cases, e.g., where multiple zones are flowing, the relative concentrations of these photoactive materials from quantitative analysis in the fluid may illustrate an estimate of the relative amount of fluid flowing from each zone.

In one embodiment, the present invention provides a method of detecting particulate or fluid flow from a first location to a second location comprising the steps of: introducing a tracer matrix comprising a polymeric material and a photoactive material to the first location, and detecting the photoactive material at the second location.

In another embodiment, the present invention provides a method of detecting flow from a first zone and a second zone in a multizonal well in a subterranean formation comprising the steps of: introducing a first photoactive tracer into the first zone; introducing a second photoactive tracer into the second zone; and detecting the first and the second photoactive tracers in the return flow from the first and second zones.

In another embodiment, the present invention provides a method of detecting flow in a multiple-stage hydraulic fracturing treatment comprising a plurality of stages comprising the steps of: introducing a photoactive tracer into each stage of the multiple stage hydraulic fracturing treatment; and detecting the photoactive tracer on a return flow.

In another embodiment, the present invention provides a method of verifying the functioning of a limiting tool that limits or restricts the flow of a fluid or particulate from a first location neighboring the limiting tool to a second location comprising the steps of: introducing a photoactive tracer into the first location neighboring the limiting tool; and detecting the photoactive tracer at the second location.

In another embodiment, the present invention provides a tracer matrix composition comprising a photoactive material and a polymeric material.

In another embodiment, the present invention provides a method of making a tracer matrix that comprises a photoactive material and a polymeric material comprising the steps of: swelling the polymeric material in an organic solvent comprising a photoactive material; and removing the solvent so as to produce a tracer matrix comprising the photoactive material and the polymeric material.

To facilitate a better understanding of the present invention, the following examples of preferred embodiments are given. In no way should such examples be read to limit the scope of the invention.

### EXAMPLES

### Example 1

500 ppm of each of the fluorophores fluorescein, rhodamine B, Nile Blue A, and acridine orange were evaluated at 325° F. in WG-18 (30 pounds per thousand gallons carboxymethyl hydroxypropyl guar, “CMHPG”), cross-linked with a zirconium crosslinker (0.3 gallons per thousand), and an alkaline hypochlorous acid breaker (0.25 gallons per thousand). Additional additives were an acetate buffer (0.05 gallons per thousand) a carbonate buffer (1.5 gallons per thousand) and an ammonium salt (2 gallons per thousand). Each fluorophore showed activity when exposed to short wave (254 nm) light after 6 days in a closed cell. At lower concentrations of fluoroscein, no fluorescent activity was observed, suggesting a possible sensitivity to alkaline hypochlorous acid until the oxidizer is spent. Breaking of the cross-linked fluid was observed by the presence of a brown precipitate and significant loss in viscosity. The viscosity was that of water after the testing.

### Example 2

In a fluid composition mimicking an actual acid treatment recipe for a high-rate water pack, fluorescein showed stability at 155° C. for 72 hours. The stability of fluorescein was tested in several ways. Each individual fluid stage was prepared and the fluorescein was added. The final solution was mixed together in ratios consistent with each individual fluid stage formulation, under the assumption that each stage completely mixed and the fluorescein was added to see if it would be affected. Finally, liquid gel concentrates made from either diesel or Arcosolv DPM containing fluorescein were added to verify that delivery of the fluorophore through these methods would not affect the stability. Each of the chemicals listed in the following formulations is available from Halliburton Energy Services in Duncan, Okla.

Fluid stage I was prepared by adding the following concentrations to water: 250 lbs/1000 gal of a clay control cationic polymer known as “CLAY-FIX II” was added to 3 gal/1000 gal of another clay control cationic polymer known as “CLA-STA,” 3 gal/1000 gal, a nonionic surfactant known as “LO-SURF” 300, 50 gal/1000 gal glycol ether for property control, and the remaining volume was tap water.

Fluid stage II was comprised of 288 gal/1000 gal 20 Baumé HCl, 10 gal/1000 gal of “Fe-1A,” “Fe2” (50 lbs/1000 gal), 2 gal/1000 gal “HAI-85M,” 15 lbs/1000 gal “Fer-chek” (each of these chemicals is for iron control), 3 gal/1000 gal “LoSURF-300” and 50 gal/1000 gal of “Musol”.

Fluid stage III was prepared by adding “K-SPAR” (an acidizing formulation comprised of 286 gal/1000 gal 20 Baumé HCI, 30 gal/1000 gal “Fe-1A,” 10 gal/1000 gal “CLA-STA FS,” 2 gal/1000 gal “PEN-88” and 133 lbs/1000 gal ammonium bifluoride) to 2 gal/1000 gal of “HAI-85M,” 50 gal/1000 gal “Musol,” 3 gal/1000 gal “LoSurf-300” and 15 lbs/1000 gal “Ferchek.”

Finally, fluid stage IV comprised 250 lbs/1000 gal “Clay-FIX(II)” 11.4 gal/1000 gal hydroxyethyl cellulose liquid, and 3 lbs/1000 gal of sodium persulfate breaker.

The results demonstrate feasibility of marking different fracturing fluid stages with different photoactive materials. While qualitative determination of fluorophore presence may be made visually or with a short-wave ultraviolet (preferably with a wavelength of about 254 nm) lamp, field determination of fluorescein concentration was quantitatively demonstrated using the HACH DR/890 field colorimeter.

### Example 3

To illustrate that the tracers of the present invention could present minimal environmental problems, a liquid gel concentrate was prepared using a surfactant (“DOC-3Z”), an acrylate suspending agent (“GS-5”), and a glycol ether solvent (“Arcosolv DPM”) or diesel in the following percentages (w/w %): 0.18%, 3.2%, and 60.2%. All of these chemicals are available from Halliburton Energy Services. This mixture was sheared at the maximum velocity of an overhead stirrer then transferred to a 45.4 g of fluorescein and sheared until homogenized. The viscosity of this mixture after 3 days without visual separation was 3500 cP as measured by a Brookfield viscometer at 20 rpm.

### Example 4

In another example, each of the resins was weighed out at 0.5 g and added to 5 mL of dioxane or tetrahydrofuran. Each sample swelled over 3 hours before 0.03 g of solid sodium fluorescein was added and thoroughly stirred into the solution to form a heterogeneous mixture. The organic solvent was carefully decanted and the resin was added to a saturated brine solution. The high ionic strength brine reversed the swelling of the resin, resulting in the fluorescein being trapped within the polymeric matrix. After 72 hours of soaking in distilled water, no evidence of leaching of the fluorophore was observed into the water from any of the samples, as observed by the use of a short wave/long wave UV lamp. The results demonstrate hydrophobic adsorption of the photoactive material into the interior of a polymer, protecting the photoactive material from bulk solution, without requiring a bond-forming reaction. The results illustrate that this technique may be used to make a viable tracer material for use in oil/gas wells.

### Example 5

In another example, Texas Red (5 mg, sulforhodamine 101 acid chloride) was added to 1 g of an amine terminated Janda Jel, swollen in methylene chloride, and the mixture was stirred overnight. Filtration, followed by washing the insoluble, tagged polymer with water and a variety of organic solvents, demonstrated that a chemical bond formed between the gel and the fluorophore. The photoactive material responded to stimulation of a short wave/long wave UV lamp demonstrating that the photoactivity of the material was not lost due to the chemical reaction. The results illustrate that this is a viable tracer for use in oil/gas wells.

Thus, the present invention is well adapted to carry out the objects and attain the ends and advantages mentioned as well as those that are inherent therein. While numerous changes may be made by those skilled in the art, such changes are encompassed within the spirit and scope of this invention as defined by the appended claims.

1. A method of detecting particulate or fluid flow from a first location to a second location comprising the steps of:
introducing a tracer matrix comprising a polymeric material and a photoactive material to the first location; and
detecting the photoactive material at the second location.

2. The method of claim 1 wherein the photoactive material comprises a fluorophore, a dye, or a pigment.

3. The method of claim 1 wherein the photoactive material comprises a fluorophore, dye, or pigment that has a blue, green, yellow, orange, orange-red, or red-far red absorption or emission spectrum.

4. The method of claim 1 wherein the polymeric material comprises a cross-linked polystyrene derivative.

5. The method of claim 1 wherein the polymeric material protects the photoactive material from degradation downhole.

6. The method of claim 1 wherein the polymeric material is substantially water-insoluble.

7. The method of claim 1 wherein the polymeric material comprises a latex, a polystyrene, a polyvinyl chloride, a polyester, a polyolefin, a polycarbonate, or a polybutadiene.

8. The method of claim 1 wherein the tracer matrix is covalently derivatized.

9. The method of claim 1 wherein the tracer matrix is formed by a nucelophilic substitution reaction, a hydroboration reaction, an organo-metallic bond-forming reaction, a pericyclic bond-forming reaction, or a combination of oxidation and reduction reactions.

10. The method of claim 1 wherein the tracer matrix is formed by an emulsion polymerization process.

11. The method of claim 1 wherein the tracer matrix is formed by coating the polymeric material on the photoactive material.

12. The method of claim 1 wherein the tracer matrix is formed by a swelling/shrinking process.

13. The method of claim 1 wherein the polymeric material protects about 50% to 100% of the surface area of the photoactive material.

14. The method of claim 1 wherein the photoactive material is embedded within the polymeric material.

15. The method of claim 1 further comprising the step of adding the tracer matrix to a fluid before introducing the matrix tracer into the first location.

16. The method of claim 1 wherein the photoactive material comprises fluorescein, rhodamine B, Nile Blue A, or acridine orange.

17. The method of claim 1 wherein the tracer matrix further comprises a second photoactive material.

18. The method of claim 1 wherein detecting the tracer at the second location comprises using a UV detector, a calorimeter, or a fluorimeter.

19. The method of claim 1 wherein detecting the tracer at the second location comprises quantitative analysis of the tracer.

20. The method of claim 1 wherein detecting the tracer at the second location comprises qualitative analysis of the tracer.

21. A method of detecting flow from a first zone and a second zone in a multizonal well in a subterranean formation comprising the steps of:
introducing a first photoactive tracer into the first zone;
introducing a second photoactive tracer into the second zone; and
detecting the first and the second photoactive tracers in the return flow from the first and second zones.

22. The method of claim 21 wherein the first photoactive tracer and the second photoactive tracer have a different absorption or emitting wavelengths.

23. The method of claim 21 wherein the first photoactive tracer or the second photoactive tracer comprises fluorescein, rhodamine B, Nile Blue A, or acridine orange.

24. The method of claim 21 wherein the first photoactive tracer or the second photoactive tracer comprises a fluorescein gel concentrate.

25. The method of claim 21 wherein the first photoactive tracer or the second photoactive tracer comprises a tracer matrix that comprises a photoactive material and a polymeric material.

26. The method of claim 25 wherein the photoactive material comprises a fluorophore, a dye, or a pigment.

27. The method of claim 25 wherein the photoactive material comprises a fluorophore, dye, or pigment that has a blue, green, yellow, orange, orange-red, or red-far red absorption or emission spectrum.

28. The method of claim 25 wherein the polymeric material protects the photoactive material from degradation downhole.

29. The method of claim 25 wherein the polymeric material is substantially water-insoluble.

30. The method of claim 25 wherein the polymeric material comprises a latex, a polystyrene, a polyvinyl chloride, a polyester, a polyolefin, a polycarbonate, or a polybutadiene.

31. The method of claim 25 wherein the tracer matrix is covalently derivatized.

32. The method of claim 25 wherein the tracer matrix is formed by a nucelophilic substitution reaction, a hydroboration reaction, an organo-metallic bond-forming reaction, a pericyclic bond-forming reaction, or a combination of oxidation and reduction reactions.

33. The method of claim 25 wherein the tracer matrix is formed by an emulsion polymerization process.

34. The method of claim 25 wherein the tracer matrix is formed by coating the polymeric material on the photoactive material.

35. The method of claim 25 wherein the tracer matrix is formed by a swelling/shrinking process.

36. The method of claim 25 wherein the polymeric material protects about 50% to 100% of the surface area of the photoactive material.

37. The method of claim 25 wherein the photoactive material is embedded within the polymeric material.

38. The method of claim 25 wherein the tracer matrix further comprises a second photoactive material.

39. The method of claim 21 wherein detecting either the first photoactive tracer or the second photoactive tracer comprises using a UV detector, a calorimeter, or a fluorimeter.

40. A method of detecting flow in a multiple-stage hydraulic fracturing treatment comprising a plurality of stages comprising the steps of:
introducing a photoactive tracer into each stage of the multiple-stage hydraulic fracturing treatment; and
detecting the photoactive tracer on a return flow.

41. The method of claim 40 wherein the photoactive tracer comprises fluorescein, rhodamine B, Nile Blue A, or acridine orange.

42. The method of claim 40 wherein the photoactive tracer comprises a tracer matrix that comprises a photoactive material and a polymeric material.

43. The method of claim 40 wherein a different photoactive tracer is introduced into each stage of the multiple-stage hydraulic fracturing treatment.

44. A method of verifying the functioning of a limiting tool that limits or restricts the flow of a fluid or particulate from a first location neighboring the limiting tool to a second location comprising the steps of:
introducing a photoactive tracer into the first location neighboring the limiting tool; and
detecting the photoactive tracer at the second location.

45. The method of claim 40 wherein the photoactive tracer comprises fluorescein, rhodamine B, Nile Blue A, or acridine orange.

46. The method of claim 40 wherein the photoactive tracer comprises a tracer matrix that comprises a photoactive material and a polymeric material.

47. A tracer matrix composition comprising a photoactive material and a polymeric material.

48. The composition of claim 47 wherein the photoactive material comprises a fluorophore, a dye, or a pigment.

49. The composition of claim 47 wherein the photoactive material comprises a fluorophore, dye, or pigment that has a blue, green, yellow, orange, orange-red, or red-far red absorption or emission spectrum.

50. The composition of claim 47 wherein the photoactive material comprises a fluorescein gel concentrate.

51. The composition of claim 47 wherein the polymeric material protects the photoactive material from degradation downhole.

52. The composition of claim 47 wherein the polymeric material is substantially water-insoluble.

53. The composition of claim 47 wherein the polymeric material comprises a latex, a polystyrene, a polyvinyl chloride, a polyester, a polyolefin, a polycarbonate, or a polybutadiene.

54. The composition of claim 47 wherein the tracer matrix is covalently derivatized.

55. The composition of claim 47 wherein the tracer matrix is formed by a nucelophilic substitution reaction, a hydroboration reaction, an organo-metallic bond-forming reaction, a pericyclic bond-forming reaction, or a combination of oxidation and reduction reactions.

56. The composition of claim 47 wherein the tracer matrix is formed by an emulsion polymerization process.

57. The composition of claim 47 wherein the tracer matrix is formed by coating the polymeric material on the photoactive material.

58. The composition of claim 47 wherein the tracer matrix is formed by a swelling/shrinking process.

59. The composition of claim 47 wherein the polymeric material protects about 50% to 100% of the surface area of the photoactive material.

60. The composition of claim 47 wherein the photoactive material is embedded within the polymeric material.

61. The composition of claim 47 wherein the photoactive material comprises fluorescein, rhodamine B, Nile Blue A, or acridine orange.

62. The composition of claim 47 wherein the tracer matrix further comprises a second photoactive material.

63. A method of making a tracer matrix that comprises a photoactive material and a polymeric material comprising the steps of:
swelling a polymeric material in an organic solvent comprising a photoactive material; and
removing the solvent so as to produce a tracer matrix comprising the photoactive material and the polymeric material.
